(19) United States
(12) Patent Application Publication
(10) Pub. No.: US 2021/0198611 A1
(43) Pub. Date: Jul. 1, 2021

(54) METHOD FOR CONDITIONING pH SENSOR, CULTURE APPARATUS, AND CULTURE CONTROL APPARATUS

(71) Applicant: ABLE Corporation, Tokyo (JP)

(72) Inventor: Shutaro ISHIKAWA, Tokyo (JP)

(21) Appl. No.: 17/058,793

(22) PCT Filed: May 10, 2019
(86) PCT No.: PCT/JP2019/019596

§ 371 Date: Nov. 25, 2020

(30) Foreign Application Priority Data
May 28, 2018 (JP) 2018112312

(51) Int. Cl. C12M 1/34; C12M 1/06; C12M 1/00

(52) U.S. Cl. CPC C12M 41/34; C12M 27/02; C12M 29/20; C12M 41/06; C12M 23/38

(57) ABSTRACT

A culturing process according to the present embodiment includes: a step of carrying out the setting of a culture apparatus 1 (Step S100); a step of carrying out the conditioning of a pH sensor 40 (Step S200); and an actual culturing step (Step S300). More specifically, in the process, the setting of the culture apparatus 1 (Step S100) is carried out; and, after the completion of the preceding step, the step of carrying out the conditioning of the pH sensor 40 (Step S200) is carried out; and subsequently the actual culturing step is carried out using the culture apparatus 1 (Step S300).

### TECHNICAL FIELD

The present invention relates to a pH sensor conditioning method, a culture apparatus, and a culture control apparatus.

### BACKGROUND ART

In a technique for culturing cells, microorganisms, etc. using a culture medium, a technique for performing a culture process while controlling the pH of the culture medium is known. For example, Patent Document 1 discloses a technique in which pH in a culture tank is measured by a pH meter, and a pH adjusting agent is charged so as to eliminate a difference between a preset pH setting value and the measured value.

### PRIOR ART DOCUMENT

### Patent Document

Patent Document 1:

Japanese Patent Application Publication S61-224982

### SUMMARY OF THE INVENTION

### Problem to be Solved by the Invention

There are various methods for measuring the pH of a liquid, and the optimum method is applied according to the purpose. In the culture field, since it is preferable to minimize the influence on a culture object and to enable continuous measurement, a diaphragm type pH sensor (pH electrode) or a pH sensor using a fluorescent dye is used.

Generally, these pH sensors are immersed in a culture medium for a certain period (from a few hours to a few days) (conditioning) in order to stabilize the output. While the output of the pH sensor is unstable during conditioning, it is preferable to adjust the pH of the culture medium to a certain range during the period.

One object of the present invention is to provide a pH sensor conditioning method, a culture apparatus, and a culture control apparatus capable of controlling the pH of a culture medium with required accuracy during a period when the output of the pH sensor is not stable.

### Means for Solving the Problem

The pH sensor conditioning method according to this invention includes the step of conditioning a pH sensor by immersing the pH sensor in a culture medium while adjusting the supply amount of carbon dioxide to the culture medium based on the measured value of dissolved oxygen of the culture medium.

In the pH sensor conditioning method, the pH sensor may be conditioned while supplying air to the culture medium.

In the pH sensor conditioning method, the pH sensor may be conditioned while adjusting the temperature of the culture medium.

The culture apparatus of this invention includes a dissolved oxygen sensor for measuring the dissolved oxygen concentration of the culture medium, a carbon dioxide gas supply module for supplying carbon dioxide gas to the culture medium, and a control module for controlling the operation of the carbon dioxide gas supply module, where the control module is configured to control the operation of the carbon dioxide gas supply module based on the measured value of the dissolved oxygen sensor.

The culture control apparatus of this invention controls the operation of the culture apparatus, and controls the operation of a carbon dioxide gas supply module for supplying carbon dioxide gas to the culture medium based on dissolved oxygen concentration information of the culture medium.

### Effect of the Invention

In this embodiment, during conditioning of the pH sensor, the supply amount of carbon dioxide gas to the culture medium is controlled based on the measured value of dissolved oxygen. This allows the pH of the culture medium to be controlled within a certain range during conditioning of the pH sensor.

The supply amount of carbon dioxide is adjusted based on the measured value of the dissolved oxygen sensor of the culture medium. Therefore, even when it is difficult to accurately measure the pH of the culture medium by the pH sensor, such as during conditioning of the pH sensor, the pH of the culture medium can be adjusted within a predetermined range.

### BRIEF DESCRIPTION OF THE DRAWINGS

FIG. 1 is a diagram for explaining a culture apparatus according to this embodiment.

FIG. 2 is a diagram for explaining the culture step according to this embodiment.

FIG. 3 is a diagram for explaining the culture step according to this embodiment.

FIG. 4 is a diagram for explaining the culture step according to this embodiment.

### MODE FOR CARRYING OUT THE INVENTION

Embodiments to which the present invention is applied will be described below. However, the present invention is not limited to the following embodiments. Namely, not all the configurations described in the following embodiments are essential to the present invention. The present invention also includes any combination of the following details.

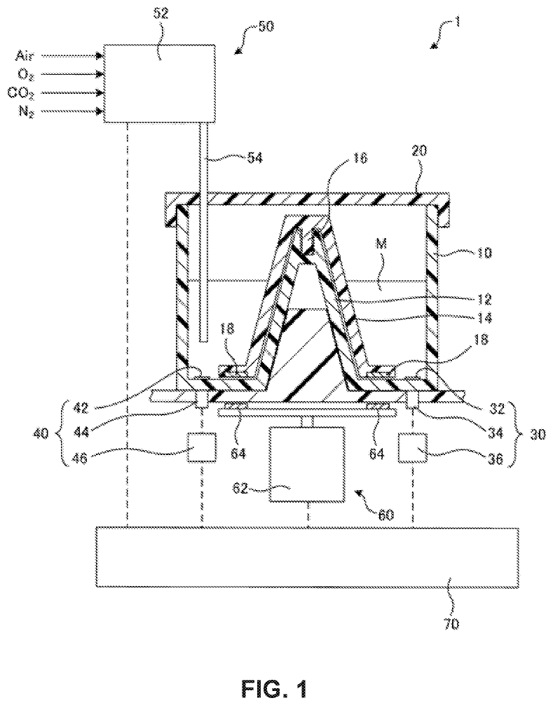

First, a configuration of a culture apparatus 1 to which the present invention can be applied will be described with reference to FIG. 1.

The culture apparatus 1 includes a culture tank 10. The culture tank 10 is a member for holding a culture medium M and a culture object, and the culture object is cultured in the culture tank 10.

The culture tank 10 is preferably made of a material having no toxicity to the culture medium and the culture object, and may be made of polycarbonate, for example.

The culture tank 10 includes a convex portion 12. The convex portion 12 is a member protruding from a bottom surface of the culture tank 10 and serves to support an agitation blade 14 described later.

The culture tank 10 include an agitation blade 14. The agitation blade 14 has a shaft 16, and the shaft 16 is inserted into a recess provided in an upper surface of the convex portion 12. Thus, the agitation blade 14 can rotate about the shaft 16. The agitation blade 14 has a magnet 18. The magnet 18 rotationally drives the agitation blade 14.

The culture tank 10 has a lid body 20. The lid body 20 is a member for closing an upper end of the culture tank 10. The lid body 20 can prevent evaporation of the culture medium M from the inside of the culture tank 10 and contamination of the culture medium M with foreign matters.

The culture apparatus 1 has a dissolved oxygen sensor 30. The dissolved oxygen sensor 30 serves to measure the dissolved oxygen concentration of the culture medium M. Any sensor that can be used for culture can be used as the dissolved oxygen sensor 30.

In this embodiment, an example is given where a sensor using a fluorescent dye is applied as the dissolved oxygen sensor 30. Namely, the dissolved oxygen sensor 30 includes a phosphor 32. The phosphor 32 is arranged inside the culture tank 10 and comes into contact with the culture medium M. In this embodiment, the phosphor 32 is attached to a bottom surface of the culture tank 10. The dissolved oxygen sensor 30 has a light receiving and emitting module 34. The light receiving and emitting module 34 can be realized by a light receiving element and a light emitting element. The light emitting element serves to irradiate the phosphor 32 with excitation light. The phosphor 32 receives excitation light to excite, and emits fluorescence when returning to a ground state. The light receiving element receives the fluorescence emitted by the phosphor 32 for conversion into an electric signal.

The phosphor 32 is adjusted so as to change the property of the emitted fluorescence based on the dissolved oxygen concentration of the culture medium M. Therefore, the dissolved oxygen concentration of the culture medium M can be measured by utilizing the information of the excitation light obtained by the light receiving element and the information of the excitation light emitted from the light emitting element.

In this embodiment, the dissolved oxygen sensor 30 includes an amplifier 36. The amplifier 36 performs necessary calculations based on the light emission information of the light receiving and emitting module 34 and the information of the excitation light from the light receiving and emitting module 34, and transmits the result to a control unit 70 (to be mentioned below). Thus, the control unit 70 can acquire the output information of the dissolved oxygen sensor 30 and control the operation of the culture apparatus 1 based on the output information.

The culture apparatus 1 has a pH sensor 40. The pH sensor 40 serves to measure the pH of the culture medium M. As the pH sensor 40, any sensor that can be used for culture can be used.

In this embodiment, an example is given where a sensor using a fluorescent dye is applied as the pH sensor 40, as in the case of the dissolved oxygen sensor 30. Namely, the pH sensor 40 has a phosphor 42 disposed inside the culture tank 10 and in contact with the culture medium M. The pH sensor 40 also includes a light receiving and emitting module 44. The light receiving and emitting module 44 has a light emitting element for emitting excitation light toward the phosphor 42 and a light receiving element for receiving the fluorescence of the phosphor 42. The light receiving element receives the fluorescence of the phosphor 42 for conversion into an electric signal.

The phosphor 42 is adjusted so that the property of the emitted fluorescence changes based on the pH of the culture medium M. Therefore, the pH of the culture medium M can be measured by using the information on the excitation light obtained by the light receiving element and the information on the excitation light emitted from the light emitting element.

In this embodiment, the pH sensor 40 includes an amplifier 46. The amplifier 46 performs necessary calculations based on the light emission information of the light receiving and emitting module 44 and the information of the excitation light from the light receiving and emitting module 44, and transmits the result to a control unit 70 (to be mentioned below). Thus, the control unit 70 acquires the output information of the pH sensor 40 and can control the operation of the culture apparatus 1 based on the output information.

Although not particularly shown, the culture apparatus 1 may be configured to have various sensors other than the dissolved oxygen sensor 30 and the pH sensor 40. As a sensor applicable to the culture apparatus 1, a sensor for measuring the concentration of dissolved carbon dioxide in the culture medium M, temperature, and turbidity can be exemplified. As described above, in the culture apparatus 1, a sensor using fluorescence is exemplified as the dissolved oxygen sensor 30 and the pH sensor 40. However, the present invention is not limited thereto, and so-called diaphragm type (electrode type) sensors can also be applied.

The culture apparatus 1 has a ventilation module 50. The ventilation module 50 serves to aseptically supply gas to the culture medium M (culture tank 10). The ventilation module 50 can be configured, for example, to supply gas such as air, oxygen, carbon dioxide, and nitrogen (and mixtures thereof) into the culture medium M. The ventilation module 50 may be configured such that the gas is introduced into the culture medium M and bubbled, or introduced into the culture tank 10 and supplied to the culture medium M through the liquid surface.

In this embodiment, the ventilation module 50 has a gas adjusting module 52. The gas adjusting module 52 serves to receive the gas supplied from a cylinder, etc. and adjust the component and amount of gas to be introduced into the culture medium M. Gas adjusted by the gas adjusting module 52 is introduced into the culture medium M through an introduction pipe 54. Namely, the amount of gas supplied to the gas adjusting module 52 is adjusted for each component and introduced into the culture medium M through the introduction pipe 54.

Although not particularly shown, the culture apparatus 1 can be configured to include various other mechanisms. The culture apparatus 1 may further include, for example, a medium supply module for supplying the medium and a sampling module for sampling the culture medium M.

The culture apparatus 1 has a driving module 60. The driving module 60 serves to drive the agitation blade 14. In this embodiment, the driving module 60 includes a magnet 62 and a motor 64 for rotating the magnet 62. The magnet 62 herein is arranged so as to face the magnet 18 of the agitation blade 14 and attract each other. Therefore, by rotating the magnet 62 by the motor 64, the agitation blade 14 can be rotationally driven about the convex portion 12.

The culture apparatus 1 has a control module 70. The control module 70 serves to integrally control the operation of the culture apparatus 1.

The control module 70 may be configured, for example, to control the operation of the gas adjusting module 52 and the motor 62. The operation of the gas adjusting module 52 and the motor 62 can be controlled based on the information of the culture medium M obtained by various sensors and the set value information of the culture medium M. Thus, since the components and amount of gas dissolved in the culture medium M are adjusted, the state of the culture medium M can be brought close to the set value. The control module 70 may be configured to adjust the temperature of the culture medium M.

The control module 70 may be configured to select a mode of operation of the culture apparatus 1. Namely, as will be described later, the culture apparatus 1 in this embodiment performs the step of conditioning the pH sensor 40 before the actual culture step. Therefore, a conditioning mode for controlling the conditioning step of the pH sensor 40 and an actual culture mode for controlling the actual culture step can be switched. During conditioning of the pH sensor 40, the control module 70 can be configured to control the operation of the gas supply module 52 (carbon dioxide gas supply module) based on the measurement value of the dissolved oxygen sensor 30.

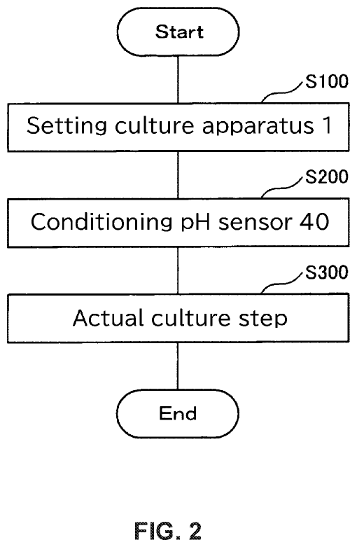

Next, a culture step using the culture apparatus 1 will be described. FIGS. 2 to 4 are flowcharts for explaining the culture step using the culture apparatus 1.

First, an outline of the culture step will be described with reference to FIG. 2. The culture step according to this embodiment includes the step of setting a culture apparatus 1 (Step S100), the step of conditioning a pH sensor 40 (Step S200), and the step of an actual culture (Step S300). Each of the steps will be described in detail below.

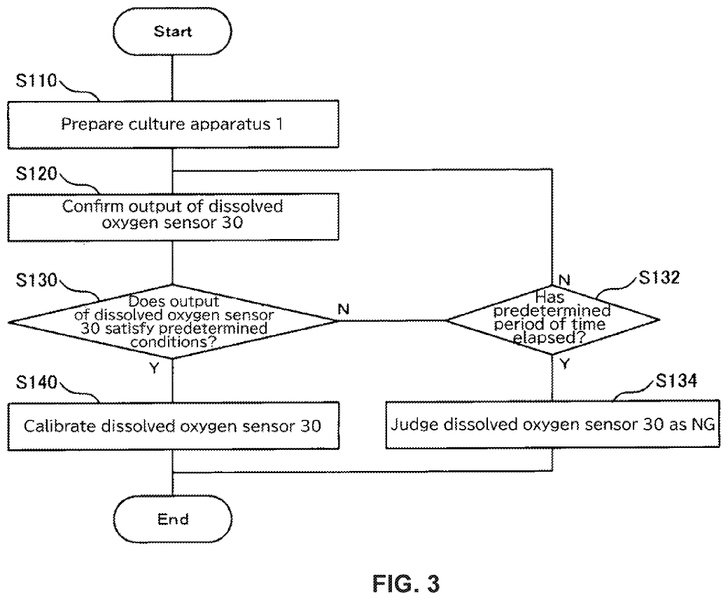

FIG. 3 is a flowchart for explaining the step of setting the culture apparatus 1 (Step S100).

This step includes the step of preparing the culture apparatus 1 (Step S110). In this embodiment, the culture medium is injected into the culture tank 10, the lid body 20 is attached, and the sensor, etc. is attached if necessary, and is positioned with respect to the driving module 60. Thus, the culture apparatus 1 is set and the driving can be started.

This step includes the step of confirming the output of the dissolved oxygen sensor 30 (Step S120). Specifically, the temperature of the culture medium M is controlled while agitating, and the output of the dissolved oxygen sensor 30 is checked while introducing air into the culture tank 10.

This step includes the step of determining whether the output of the dissolved oxygen sensor 30 measured in Step S120 satisfies a predetermined condition (Step S130). Namely, normally, when air is introduced into the culture tank 10, oxygen in the air is dissolved in the culture medium M, and the dissolved oxygen concentration in the culture medium M gradually approaches a constant value. Therefore, if the output of the dissolved oxygen sensor 30 reaches a constant value within a predetermined period (Yin Step S130), the dissolved oxygen sensor 30 is calibrated by setting the value as 100% saturation (Step S140).

However, if the output of the dissolved oxygen sensor 30 is unstable even after a predetermined period of time has elapsed (N in Step S130, and Yin Step S132), there is a possibility of some defect in the culture apparatus 1, so that the culture apparatus 1 is judged as NG (Step S134), and the step is terminated.

By the above steps, the setting of the culture apparatus 1 (Step S100) is completed.

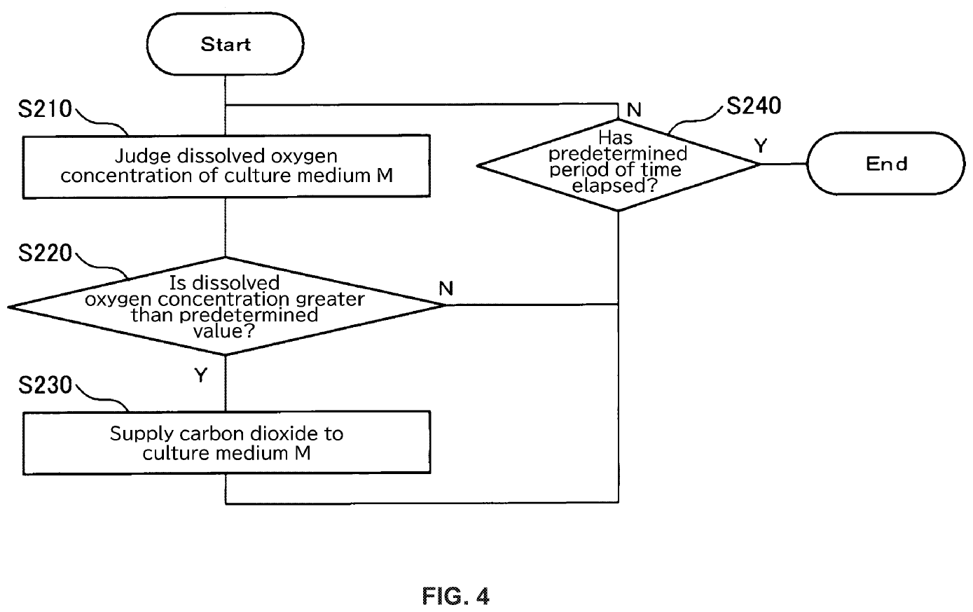

FIG. 4 is a diagram for explaining the step of conditioning the pH sensor 40 (Step S200).

This step includes the step of measuring the dissolved oxygen concentration of the culture medium M (Step S210). In this embodiment, the dissolved oxygen concentration in the culture medium M can be measured by the dissolved oxygen sensor 30 while agitating the culture medium M while controlling the temperature and introducing air into the culture tank 10 (culture medium M). This step includes the step of determining whether the dissolved oxygen concentration of the culture medium M is greater than a predetermined value (Step S220). When the dissolved oxygen concentration of the culture medium M is greater than a predetermined value (Y in Step S220), carbon dioxide is supplied to the culture medium M (Step S230). When the dissolved oxygen concentration of the culture medium M is less than a predetermined value (N in Step S220), carbon dioxide is not supplied to the culture medium M. Namely, in this embodiment, the amount of carbon dioxide to be supplied to the culture medium M is adjusted based on the dissolved oxygen concentration of the culture medium M. This step is repeated until a predetermined time has elapsed (until Y in Step S240) to complete this step. Thus, since the pH sensor 40 is adapted to the culture medium M and the output of the pH sensor 40 is stabilized, the pH of the culture medium M can be accurately measured.

Thereafter, the step of actual culture is performed by using the culture apparatus 1 (Step S300). In this step, the culture medium M is changed as necessary, the pH sensor 40 is calibrated, and a culture object such as a cell is seeded in the culture tank 10, and the culture object is cultured. In this step, the culture medium M is agitated while controlling the temperature, and the supply amounts of oxygen and carbon dioxide to the culture medium M are adjusted according to the outputs of the dissolved oxygen sensor 30 and the pH sensor 40. Thus, the state of the culture medium M can be precisely controlled, and the culture of the culture object can be appropriately controlled.

The culture process is terminated by culturing the culture object for a predetermined period of time while adjusting the state of the culture medium M.

However, as a modification, it is also possible to perform setting of the culture apparatus 1 and conditioning of the pH sensor 40 while the culture medium M is seeded with the culture object. When the activity of the culture object is low, even if the culture object is contained in the culture medium M, the pH of the culture medium M can be controlled using the dissolved oxygen concentration 30 since the dissolved oxygen concentration of the culture medium M has a certain correlation with the pH.

Next, the functional effect of this embodiment will be described.

As described above, in this embodiment, the amount of carbon dioxide supplied to the culture medium M is adjusted based on the dissolved oxygen concentration during the conditioning step of the pH sensor 40. Namely, the supply of carbon dioxide to the culture medium M is controlled so that the dissolved oxygen concentration of the culture medium M reaches a predetermined value. This allows the pH of the culture medium M to be adjusted to a predetermined range even during conditioning of the pH sensor 40.

Specifically, although the pH of the culture solution M can be adjusted by the supply amount of carbon dioxide, since the output of the pH sensor 40 is not stable during conditioning of the pH sensor 40, it is difficult to measure the pH of the culture medium M by using the pH sensor 40. Since the pH of the culture medium M correlates with the amount of carbon dioxide in the culture medium, it is possible to calculate the pH of the culture medium M based on the amount of carbon dioxide supplied to the culture medium M. For this purpose, however, it is necessary to accurately control the supply amount of carbon dioxide, and a large scale of equipment is required.

On the other hand, according to this embodiment, the pH of the culture medium M is controlled by controlling the amount of carbon dioxide supplied to the culture medium M based on the dissolved oxygen concentration of the culture medium M. Namely, when carbon dioxide is supplied while air is supplied to the culture medium M, the oxygen in the culture medium M is expelled, and the dissolved oxygen concentration decreases. On the contrary, when the supply of carbon dioxide is stopped, the oxygen in the culture medium M approaches the saturation concentration, so that the dissolved oxygen concentration increases. Namely, the dissolved oxygen concentration of the culture medium M has a certain correlation with the amount of carbon dioxide supplied to the culture medium M. Therefore, the pH of the culture medium M can be adjusted to a predetermined value by controlling the supply of carbon dioxide to the culture medium M so that the dissolved oxygen concentration of the culture medium M reaches a predetermined value.

Namely, according to this embodiment, it is possible to control the pH of the culture medium to a constant range during conditioning of the pH sensor without using a mechanism for precisely controlling the supply amount of carbon dioxide, and it is possible to prevent degeneration of the culture medium during conditioning.

According to this embodiment, stability of an output of the pH sensor can be confirmed during conditioning of the pH sensor. Namely, in this embodiment, when a predetermined time elapses in the conditioning step and the pH sensor is conditioned, the output of the pH sensor 40 will correlate with the output of the dissolved oxygen sensor 30. On the other hand, if the output of the dissolved oxygen sensor 30 does not correlate with the output of the pH sensor 40 even after a predetermined period of time has elapsed in the conditioning step, there may be a problem with the pH sensor 40 (culture apparatus 1). Therefore, according to this embodiment, it is possible to detect a malfunction of the apparatus before the culture step. By utilizing this characteristic, when it is confirmed that there is a certain correlation between the output of the pH sensor and the output of the dissolved oxygen sensor 30, it is possible to determine that the conditioning of the pH sensor has been completed.

### INDUSTRIAL APPLICABILITY

In a technique for culturing cells, microorganisms, etc. using a culture medium, a technique for performing a culture process while controlling the pH of the culture medium is known.

There are various methods for measuring the pH of a liquid, and the optimum method is applied according to the purpose. In the culture field, for example, a diaphragm type pH sensor (pH electrode) or a pH sensor using a fluorescent dye is used, and these pH sensors are generally subjected to a treatment of being immersed in a culture medium (conditioning) for a certain period (from a few hours to a few days) in order to stabilize the output.

While these sensors are unstable in the output of the pH sensor during conditioning, the present sensor is likely to be used in the field of culture since it is possible to adjust the pH of the culture medium to a certain range during such a period.

### DESCRIPTION OF REFERENCE NUMERALS

1 culture apparatus
10 culture tank
12 convex portion
14 agitation blade
18 magnet
20 lid body
30 dissolved oxygen sensor
32 phosphor
34 light receiving and emitting module
36 amplifier
40 pH sensor
42 phosphor
44 light receiving and emitting module
46 amplifier
50 ventilation module
52 gas adjusting module
54 introduction pipe
60 driving module
62 magnet
64 motor
70 control module
M culture medium

1. A method of conditioning a pH sensor comprising conditioning the pH sensor by immersing the pH sensor in a culture medium while adjusting an amount of carbon dioxide supplied to the culture medium based on a measured value of dissolved oxygen in the culture medium.

2. The method of conditioning a pH sensor according to claim 1, comprising conditioning the pH sensor while supplying air to the culture medium.

3. The method of conditioning a pH sensor according to claim 1, comprising conditioning the pH sensor while adjusting temperature of the culture medium.

4. A culture apparatus comprising:
a dissolved oxygen sensor for measuring concentration of dissolved oxygen in a culture medium;
a pH sensor for measuring pH of the culture medium;
a carbon dioxide gas supply module for supplying carbon dioxide gas to the culture medium; and
a control module for controlling operation of the carbon dioxide gas supply module,
wherein the control module is configured to switch between:
a conditioning mode to control operation of the carbon dioxide gas supply module based on measured value of the dissolved oxygen sensor while immersing the pH sensor in the culture medium for conditioning, and
an actual culture mode for controlling operation of the carbon dioxide gas supply module based on a measured value of the pH sensor after the conditioning mode.

5. A culture control apparatus for controlling operation of a culture apparatus comprising, while immersing a pH sensor for measuring pH of a culture medium in the culture medium for conditioning, controlling operation of a carbon dioxide gas supply module for supplying carbon dioxide gas to the culture medium based on dissolved oxygen concentration information of the culture medium.
